(19) United States
(12) Patent Application Publication
(10) Pub. No.: US 2004/0002130 A1
(43) Pub. Date: Jan. 1, 2004

(54) Disruption of PQS synthesis using precursor analogs

(76) Inventors: Everett C. Pesci, Greenville, NC (US); James P. Coleman, Farmville, NC (US)

Correspondence Address: MYERS BIGEL SIBLEY & SAJOVEC, PO BOX 37428, RALEIGH, NC, 27627, US

(21) Appl. No.: 10/184,218

(22) Filed: Jun. 27, 2002

(51) Int. Cl. C12Q 1/18

(52) U.S. Cl. 435/32

(57) ABSTRACT

The present invention concerns methods of detecting bacterial infections as well as methods of treating such infections with compounds such as methyl anthranilate. The detection and treatment of Pseudomonas is particularly preferred.

### STATEMENT OF FEDERAL SUPPORT

The present invention was made with Government support under Grant Number RO1-AI46682 from the National Institutes of Health. The Government has certain rights to this Invention.

### FIELD OF THE INVENTION

The present invention concerns methods of detecting PQS and bacterial cells producing the same, as well as methods of treating bacterial infections in a subject in need thereof.

### BACKGROUND OF THE INVENTION

Cell-to-cell signaling by Pseudomonas aeruginosa occurs through a complex circuitry of interconnected, regulatory systems that controls over 70 different genes (Pesci and Iglewski (1997) Trends in Micro. 5:132-135; Whiteley, et al. (1999) Proc. Nail. Acad. Sci. USA 96:13904-13909). The signaling systems of P. aeruginosa are necessary for virulence in multiple infectious disease model systems (de Kievet and Iglewski (2000) Infect. Immun. 68:4839-4848). P. aeruginosa produces two cell-to-cell signals from the acyl homoserine lactone family [N-3-oxododecanoyl)-L-homoserine lactone and N-butyryl-L-homoserine lactone], and a third, unique cell-to-cell Pseudomonas quinolone signal, referred to as PQS (Pesci, et al. (1999) Proc. Natl. Acad. Sci. USA 96:11229-11234; Pearson, et al. (1994) Proc. Nat. Acad. Sci. USA 91:197-201; Pearson, et al. (1995) Proc. Nat. Acad. Sci. USA 92:1490-1494). PQS, which induces the expression of virulence factors elastase and rhlI (encodes the N-butyryl-L-homoserine lactone synthase), is the only quinolone compound known to act as a cell-to-cell signal (Pesci, et al. (1999) Proc. Natl. Acad. Sci. USA 96:11229-11234; McKnight, et al. (2000) J. Bacteriol. 182:2702-2708). Although the role of this signal in the pathogenesis of human infections is not known, it is clearly important for P. aeruginosa virulence in a nematode killing assay (Gallagher and Manoil (2001) J. Bacteriol. 183:6207-6214). Also important to note, is that PQS signaling is regulated differently from acyl homoserine lactone signaling. Acyl homoserine lactone signals are produced at a time of rapid population growth, but PQS is produced maximally in the late stationary phase of growth (McKnight, et al. (2000) J. Bacteriol. 182:2702-2708). This suggests that PQS signaling is important when P. aeruginosa cells are under stressful conditions, such as those which would occur during a chronic infection in the lungs of cystic fibrosis (CF) patients.

As an opportunistic pathogen, P. aeruginosa has found a specialized niche in the lungs of CF patients (Gilligan (1991) Clin. Microbiol. Rev. 4:35-51). Due to poor pulmonary clearance, P. aeruginosa chronically infects the lungs of a vast majority of CF patients at a young age (Welsh, et al. In: The Metabolic and Molecular Basis of Inherited Disease Vol. III (eds. Scriver, et al.) 3799-3876 (McGraw-Hill, New York, 1995)). These infections are difficult to treat and persist for the life of the patient, causing progressive lung damage that eventually leads to respiratory failure (Koch and Hoiby (1993) Lancet 341:1065-1069). Hence, as P. aeruginosa can have devastating effects on individuals suffering from CF or other infections and can rapidly develop antibiotic resistance there is a need in the art for novel therapeutic agents to combat P. aeruginosa infections. Intercellular signals and their synthetic pathways have been suggested as starting points for novel therapeutic targets (Govan and Deretic (1996) Microbiol. Rev. 60:539-547; Calfee, et al. (2001) Proc. Natl. Acad. Sci. USA 98:11633-11637). In fact, earlier work has shown that PQS is required for virulence in an insect model of infection (Gallagher & Manoil (2001) J. Bacteriol. 183:6207-6214). In addition, the use of a PQS precursor analog was found to interfere with PQS synthesis and inhibit the production of elastase, a PQS-controlled virulence factor (Calfee, et al. (2001) Proc. Natl. Acad. Sci. USA 98:11633-11637).

### SUMMARY OF THE INVENTION

A first aspect of the present invention is a method for detecting PQS production in bacterial cells. In general, the method comprises the steps of: collecting a bacterial cell sample; culturing the cells in vitro; then collecting supernatant from said cultured cells; preparing an extract of said supernatant; and then exposing said extract to ultraviolet light, and then detecting (e.g., visually or optically detecting) blue fluorescence from said extract, wherein blue fluorescence of said extract indicates the production of PQS by said bacterial cells.

A second aspect of the invention is a method of determining the quantity of Psueodomonas present in a biological sample, comprising the steps of: providing a biological sample collected from a mammalian subject; and then detecting the quantity of PQS in said biological sample, with the quantity of PQS present in said biological sample correlated to the amount of Pseudomonas in said biological sample.

A further aspect of the present invention is a method of treating a bacterial infection (e.g., a Pseudomonas infection) in a subject in need thereof, comprising administering to said subject a compound of Formula I, or a pharmaceutically acceptable salt thereof, in an amount effective to treat said bacterial infection;

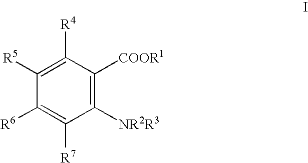

wherein:

R¹ is loweralkyl;

R² and R³ are each independently selected from the group consisting of H and loweralkyl; and

R⁴, R⁵, R⁶, and R⁷ are each independently selected from the group consisting of halo, H, and loweralkyl.

A still further aspect of the present invention is the use of a compound of Formula I above, or a pharmaceutically acceptable salt thereof, for the preparation of a medicament for carrying out a treatment as described above.

The foregoing and other objects and aspects of the present invention are explained in detail in the specification set forth below.

### BRIEF DESCRIPTION OF THE DRAWINGS

FIG. 1 shows TLC analysis of P. aeruginosa cultured from CF patients. Strain identification numbers are indicated above each lane unless otherwise indicated. PQS is indicated by an arrowhead and synthetic PQS (sPQS) has been described previously (Pesci, et al. (1999) Proc. Natl. Acad. Sci. USA 96:11229-11234).

FIG. 2 shows TLC analysis of sputum samples from CF patients infected with P. aeruginosa. Sputum sample numbers are indicated above each lane unless otherwise indicated. Control sample was from an uninfected volunteer. The relative density of P. aeruginosa in each sputum sample is indicated below each lane. PQS is indicated by an arrowhead.

FIG. 3 shows TLC analysis of bronchoalveolar lavage fluids (BALF) samples from CF patients. BALF sample numbers are indicated above each lane unless otherwise indicated. An extract from wild-type strain PAO1 is as indicated. The status of each patient with regard to P. aeruginosa infection is indicated with a “+” for positive or “−” for negative (“−” samples came from patients infected by H. influenzae). PQS is indicated by an arrowhead.

FIG. 4 shows a PQS profile of mucopurulent airway fluid from a freshly resected CF lung. Airway sample 122601KK-1 and extract of wild-type strain PAO1 are indicated. PQS is indicated by an arrowhead. The slight shift of R_{f} values for the lanes containing sample extracts was caused by exceeding the loading capacity of the TLC plate.

### DETAILED DESCRIPTION OF PREFERRED EMBODIMENTS

“PQS” as used herein refers to the Pseudomonas quinolone signal, 2-heptyl-3-hydroxy-4-luinolone. See, e.g., M. Calfee et al., Proc. Natl. Acad. Sci. USA 98, 11633-11637 (Sep. 25, 2001).

The present invention is primarily concerned with the diagnosis, screening and treatment of human subjects, but the invention may also be carried out on animal subjects, particularly mammalian subjects such as mice, rats, dogs, cats, livestock and horses for veterinary purposes, and for drug screening and drug development purposes.

Pseudomonas and Pseudomonas infection as used herein includes any type of Pseudomonas and a corresponding infection, including but not limited to Pseudomonas aeruginosa, Pseudomonas Mallei, and Pseudomonas pseudomallei, and infections therewith.

Patients with which both the methods of detection and methods of treatment of the present invention are concerned include patients afflicted with a Pseudomonas aeruginosa infection of the urinary tract, respiratory system, and soft tissue, as well as patient afflicted with dermatitis, bacteremia or other systemic infections. Horses afflicted with Pseudomonas mallei in the disease known as glanders are subjects for the present invention, as well as humans afflicted with Pseudomonas pseudomallei in the tropical disease known as melioidosis.

“Treat” as used herein with respect to bacterial infections refers to any beneficial treatment, including decreasing the virulence or infectivity of an organism, slowing the growth or proliferation of the organism, killing or reducing the population of the organism, etc.

A “biological sample” as used herein includes any suitable biological sample, including but not limited to blood samples (including blood fractions), tissue samples, sputum samples, bronchial lavage samples, urine samples, etc.

“Loweralkyl” as used herein refers to C1-C4 alkyl, which may be linear or branched, such as methyl, ethyl, propyl, and butyl.

“Halo” as used herein refers to any suitable halogen, such as fluoro, chloro bromo, and iodo.

1. Optical and Visual Methods of Detecting PQS.

As noted above, the present invention provides a method for detecting PQS production in bacterial cells. In general, the method comprises the steps of:

collecting a bacterial cell sample (e.g., a Pseudomonas sample);

culturing the cells in vitro; then

collecting supernatant from said cultured cells;

preparing an extract of said supernatant; and then

exposing said extract to ultraviolet light, and then

detecting (e.g., visually or optically detecting) blue fluorescence from said extract, wherein blue fluorescence of said extract indicates the production of PQS by said bacterial cells.

In general, the biological sample is collected from a mammalian subject. Any suitable biological sample may be collected, including but not limited to those described above. Preparation of the extract may be carried out by any suitable means, such as by extraction with acidified ethyl acetate, and then separation of the extract by a chromatographic technique such as thin layer chromatography. Detection of blue fluorescence may be carried out manually/visually, assisted with optical devices, or automatically with optical devices and/or cameras, in accordance with known techniques.

2. Methods of Screening for Pseudomonas Infection.

The present invention further provides a method of determining the quantity of Psueodomonas present in a biological sample, comprising the steps of: providing a biological sample collected from a mammalian subject; and then detecting the quantity of PQS in said biological sample, with the quantity of PQS present in said biological sample correlated (i.e., directly correlated rather than inversely correlated) to the amount of Pseudomonas in said biological sample. As above, any suitable biological sample may be employed. Also as above, the detecting step may be carried out by exposing said sample or an extract thereof to ultraviolet light and visually or optically detecting blue fluorescence of PQS in said sample.

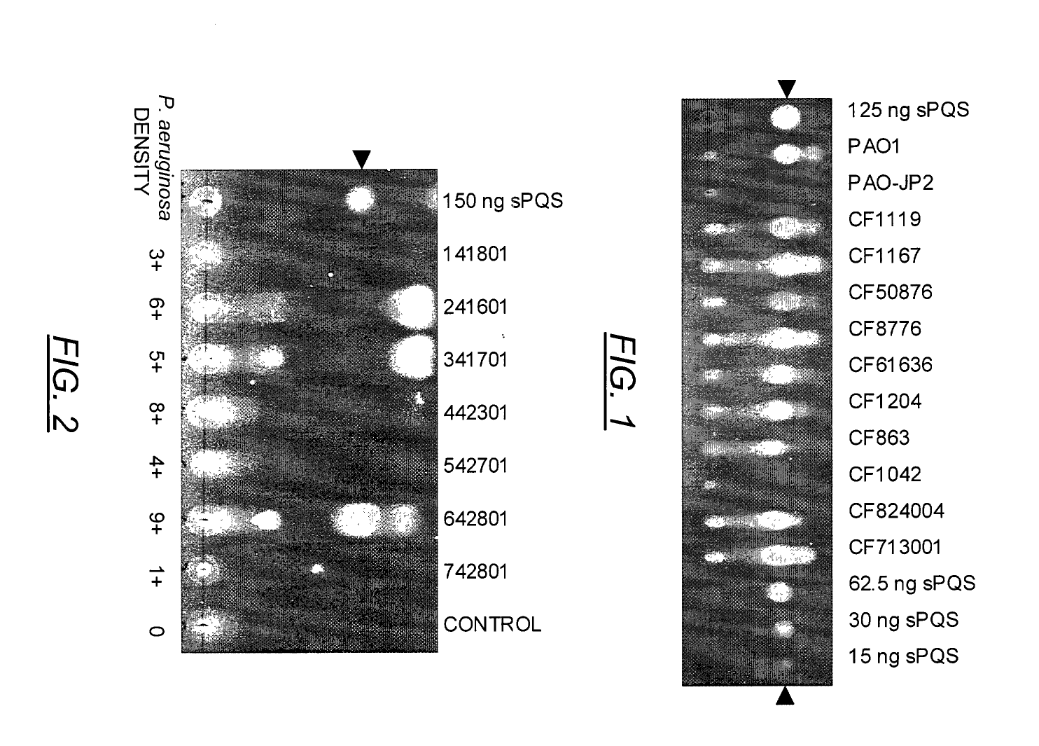

In vivo cell-to-cell signaling has been demonstrated in mice, and cell-to-cell signals are produced by Pseudomonas aeruginosa growing “ex vivo” within sputum samples from infected CF patients (Singh, et al. (2000) Nature 407:762-764; Wu, et al. (2000) Microbiology 146:2481-2493). As will be described in greater detail, it was determined that P. aeruginosa living within the CF lung produced the intercellular signal PQS. Several P. aeruginosa isolates that were cultured from CF patients were assayed for the ability to produce PQS in vitro. Culture supernatants were extracted with acidified ethyl acetate and extracts were separated by TLC. Under the solvent conditions described herein, PQS fluoresces blue under ultraviolet light and can be distinguished from other P. aeruginosa-secreted organic molecules (SOMs) found in culture extracts (Pesci, et al. (1999) Proc. Natl. Acad. Sci. USA 96:11229-11234). For the current study, a set of ten strains cultured from different patients were analyzed and it was found that nine of the ten produced varying amounts of PQS (FIG. 1). Seven of the CF strains and the prototype strain PAO1 also produced an unidentified SOM (Compound B), which fluoresced orange under ultraviolet light and had a slightly higher R_{f} value than PQS. One strain, CF1042, had a SOM profile similar to the control strain PAO-JP2 (lasI, rhlI), which did not produce detectable amounts of PQS and Compound B. These results led to the conclusion that many P. aeruginosa CF strains are capable of producing PQS when grown in vitro.

These findings indicate that each type of CF sample tested contained the cell-to-cell signal PQS. Most interestingly, the amount of PQS present in sputum samples, which are relatively simple to collect, correlated directly with the amount of P. aeruginosa in the sample. At least one other P. aeruginosa SOM was also present in some samples, indicating that SOMs may be useful as markers that could help differentiate individual infections with regard to aspects such as the degree of pathogenicity of a particular strain and/or the infectious stage that has developed. Others have shown that P. aeruginosa produces a biofilm within the CF lung and that cell-to-cell signaling is required for P. aeruginosa biofilm growth in vitro (Singh, et al. (2000) Nature 407:762-764; Davies, et al. (1998) Science 280:295-298). The demonstration of PQS in the CF lung provides evidence that this signal may be important for the development and/or maintenance of the chronic infection state. The mechanism for this has yet to be determined, but the fact that PQS was found in all three types of samples tested suggests that the in vivo production of this compound is probably benefiting P. aeruginosa in the lungs of CF patients. Finally, these results indicate that the PQS concentration in the sputum sample which contained the highest amount of PQS was approximately 2 μM. This result, accompanied by the fact that PQS was seen even in liquid as dilute as BALF, suggested that a physiologically significant amount of this signal was being produced in the lung.

3. Methods of Treatment.

17. A method of treating a bacterial infection (e.g., a Pseudomonas infection) in a subject in need thereof, comprising administering to said subject a compound of Formula I, or a pharmaceutically acceptable salt thereof, in an amount effective to treat said bacterial infection;

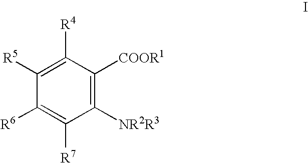

wherein:

R¹ is loweralkyl (preferably methyl);

R² and R³ are each independently selected from the group consisting of H and loweralkyl (preferably H); and

R⁴, R⁵, R⁶, and R⁷ are each independently selected from the group consisting of halo, H, and loweralkyl (preferably H).

The methods of the present invention include the administration of compounds of Formula I, while pharmaceutical compositions of the present invention comprise compounds of Formula I. As used herein, a compound of Formula I is as follows:

A particularly preferred compound of Formula I is methyl anthranilate, or 2-aminobenzoic acid methyl ester (Merck Index Reg. No. 6099, 12^{th} Edition 1996). Compounds of Formula I can be made in accordance with known techniques or variations thereof which will be apparent to those skilled in the art.

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy consisting essentially of admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising a compound of Formula (I), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to compounds of formula (I) or their salts, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration.

Preferred routes of parenteral administration include intrathecal injection, including directly into the tumor, and intraventricular injection into a ventricle of the brain.

The therapeutically effective dosage of any specific compound, the use of which is in the scope of present invention, will vary somewhat from compound to compound, and patient to patient, and will depend upon the condition of the patient and the route of delivery. For example, a dosage from about 0.1 or 0.5 to about 20 or 50 mg/kg may be used, with all weights being calculated based upon the weight of the active compound, including the cases where a salt is employed.

The examples, which follow, are set forth to illustrate the present invention, and are not to be construed as limiting thereof.

### EXAMPLE 1

### Sample Collection

All CF patient samples were collected at the Cystic Fibrosis/Pulmonary Research and Treatment Center at The University of North Carolina following an Internal Review Board-approved protocol that adheres to all federal and institutional requirements for informed consent and confidentiality. All P. aeruginosa cultures from CF patients, except for strains CF824004 and CF713001, were obtained from the University of North Carolina Hospital Clinical Microbiology Laboratory. Pure cultures of strains CF824004 and CF713001 were isolated by plating an aliquot of bronchoalveolar lavage fluids (BALF) from samples 824004 and 713001 onto Bacto Pseudomonas Isolation Agar (Difco Laboratories, Detroit, Mich.). P. aeruginosa strain PAO1 (wild-type) and PAO-JP2 (lasI, rhlI) have been described previously (Holloway, et al. (1979) Microbiol. Rev. 43:73-102; Pearson, et al. (1997) J. Bacteriol. 179:5756-5767). All strains were stored at −80° C. in 10% skim milk (McKnight, et al. (2000) J. Bacteriol. 182:2702-2708). An estimate of the density of P. aeruginosa in each sample was determined at the University of North Carolina Hospital Clinical Microbiology Laboratory by a dilution streak plate method in which a grade of 0, 1+, 2+, 3+, or 4+was assigned depending on the growth seen in each dilution streak on a plate (0=no bacteria and 4=bacteria present after 4 dilution streaks). The final P. aeruginosa density grade assigned to each sample was the sum of the density grades for each different identifiable strain of P. aeruginosa. It should be noted here that sputum samples 241601, 341701, 442301, and 642801 contained at least two different strains of P. aeruginosa and therefore received grades higher than 4+. Sputum was stored at −80° C. until analysis for PQS content. BALF samples were obtained by lavage with sterile 0.9% NaCl and stored at −80° C. before analysis. The presence of P. aeruginosa in BALF samples was determined at the University of North Carolina Hospital Clinical Microbiology Laboratory by plate culture of aliquots from each sample. Mucopurulent lung fluid was obtained from the airways of an infected CF lung that had been removed from a transplant patient. A 1.0 ml syringe was used to collect approximately 1.0 ml of mucopurulent airway fluid which was then stored at −80° C. before analysis.

### EXAMPLE 2

### TLC Methods

Bacteria from frozen stocks were grown overnight on PTSB agar plates (Ohman, et al. (1980) J. Bacteriol. 142:836-842) at 37° C. Isolated colonies were used to inoculate 10 ml of PTSB which was then incubated for 18 hr at 37° C. with vigorous shaking. Cultures were diluted 1:100 into fresh media and grown for 24 hr as above. A 400 μl aliquot of each culture was mixed with 800 μl of acidified ethyl acetate (Pesci, et al. (1999) Proc. Natl. Acad. Sci. USA 96:11229-11234), vortexed vigorously for 30 sec, then centrifuged for 10 min at 16,000×g. The organic phase was transferred to a fresh tube, and the aqueous phase was re-extracted with an additional 800 μl of solvent. The organic phases were combined, dried to completion and the solute was dissolved in 200 μl of a 1:1 mix of acidified ethyl acetate:acetonitrile. An amount of extract derived from 5 μl of culture was loaded onto the TLC plate. TLC was performed with a silica gel 60 F₂₅₄ TLC plate (EM Science) that had been soaked in 5% KH₂PO₄ and activated at 100° C. for one hour. Extracts were separated with the use of 17:2:1 methylene chloride:acetonitrile:dioxane as the solvent. When the solvent front reached the top of the plate, the plate was illuminated with UV light and photographed.

### EXAMPLE 3

### PQS in Sputum Samples from CF Patients

TLC analysis was conducted on sputum from seven infected CF patients and an uninfected, non-CF volunteer. A 400 μl aliquot of sputum was mixed with 300 μl of 0.9% NaCl, vortexed vigorously, and then extracted twice with 700 μl of acidified ethyl acetate as described above. The organic phase was dried, dissolved in 20 μl of a 1:1 mix of acidified ethyl acetate:aceonitrile, and the entire sample was loaded onto a TLC plate which was analyzed as described above.

Sputum samples from four infected individuals exhibited both PQS and compound B (FIG. 2, lanes 3, 4, 5, and 7), which indicated that PQS was produced in vivo. Two sputum samples displayed Compound B and no PQS (FIG. 2, lanes 2 and 6), and one sputum sample was similar to sputum from the uninfected volunteer, which contained no apparent P. aeruginosa SOMs (FIG. 2, lanes 8 and 9). It is interesting to note that the amount of PQS evident in each extract correlated with the relative density of P. aeruginosa in each sample. The largest amount of PQS was recovered from sputum sample 642801 (FIG. 2). When the PQS in sample 642801 was compared densitometrically to a set of PQS standards (data not shown), it was estimated that the concentration of PQS in the sputum from this patient was 2 PM. This concentration is on the edge of the physiologically active range, but it must be assumed that this is an underestimate of the actual amount because of the inefficient PQS recovery achieved by organic extraction (Pesci, et al. (1999) Proc. Natl. Acad. Sci. USA 96:11229-11234).

### EXAMPLE 4

### PQS in BALF Samples from CF Patients

Since sputum must pass through the oral cavity and consists of a mixture of lower and upper respiratory tract secretions, samples collected below the larynx were next examined. BALF from four CF patients was tested first. Two of the patients were infected with only P. aeruginosa and the other two patients were infected with only Haemophilus influenzae. A 200 μl aliquot of each BALF sample was extracted with 800 μl of acidified ethyl acetate and the entire sample was analyzed by TLC as described above.

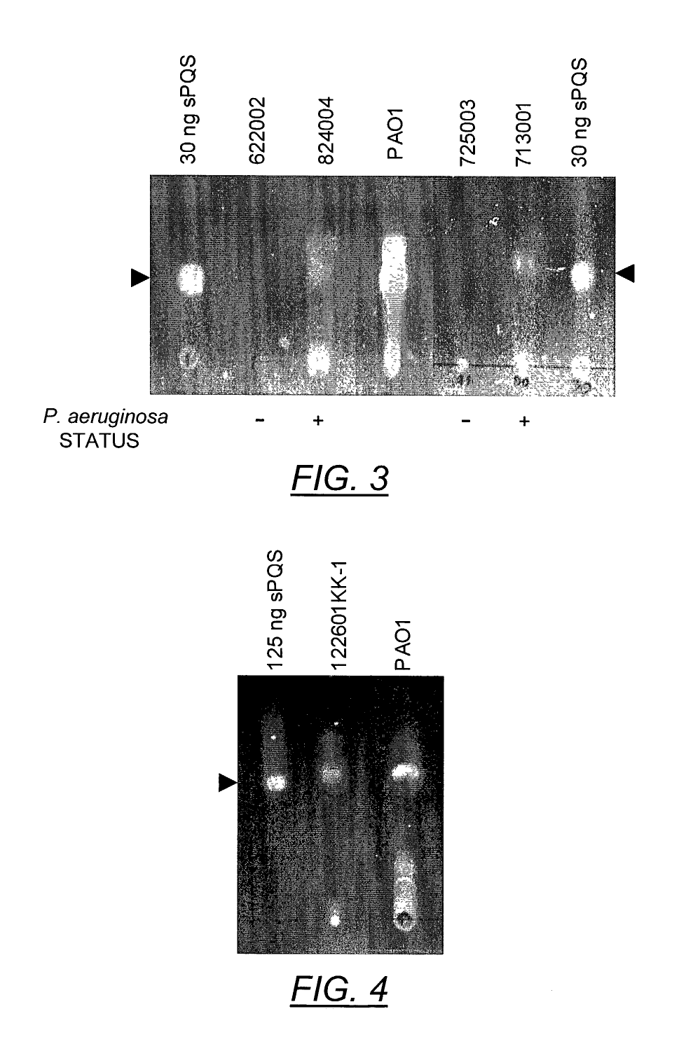

The results showed that PQS was present in the BALF samples from both patients infected by P. aeruginosa (FIG. 3). In addition, Compound B was also present in the BALF from one of these patients (FIG. 3, lane 3). The BALF control samples from the patients infected with only H. influenzae contained no PQS or Compound B (FIG. 3). These data indicate that PQS was produced by P. aeruginosa in the bronchoalveolar space of CF patients.

### EXAMPLE 5

### PQS in Mucopurulent Airway Fluid Samples from CF Patients

TLC analysis was also conducted on mucopurulent airway from two different sites within a freshly resected lung of a CF patient who was a transplant recipient infected with P. aeruginosa. Approximately 1.0 ml of supernatant from two different airway locations was mixed with 1 ml 0.9% saline, vortexed vigorously, and extracted twice with 2.0 ml acidified ethyl acetate as described above. The resultant solutes were dissolved in 60 μl of a 1:1 mix of acidified ethyl acetate:acetonitrile and the sample was centrifuged at 16,000×g for 5 min. at room temperature. The supernatant was then loaded onto a TLC plate and analyzed as described above.

Previous microscopic examination of mucopurulent airway fluid such as this showed that bacterial macrocolonies/biofilms are randomly dispersed in this fluid (Worlitzsch, et al. (2002) J, Clin. Invest. 109:317-325). The results presented herein show that both samples contained PQS and compound B (FIG. 4), which provides additional evidence that PQS is produced inside the CF lung.

### EXAMPLE 6

### Effect of Methyl Anthranilate on PQS Production

P. aeruginosa strain PAO1 was grown in the presence of increasing amounts of methyl anthranilate, and culture supernatants were extracted with ethyl acetate and separated on TLC plates. PQS was visualized under long-wave UV light and photographed to quantify PQS densitometrically. The amount of PQS present in the culture supernatant decreased as the concentration of methyl anthranilated increased, although the growth of the cultures was not affected by the presence of methyl anthranilate at the concentrations used in these experiments (0, 0.25, 0.5, 1.0, and 1.5 mM methyl anthranilate, with approximately 30% PQS being produced at 1.5 mM methyl anthranilate as compared to wild type bacateria).

The foregoing examples are illustrative of the present invention, and are not to be construed as limiting thereof. The invention is described by the following claims, with equivalents of the claims to be included therein.

What is claimed is:

1. A method for detecting PQS production in bacterial cells, comprising the steps of:
collecting a bacterial cell sample;
culturing said cells in vitro; then
collecting supernatant from said cultured cells;
preparing an extract of said supernatant; and then
exposing said extract to ultraviolet light, and then
detecting blue fluorescence from said extract, wherein blue fluorescence of said extract indicates the production of PQS by said bacterial cells.

2. The method of claim 1, wherein said bacterial cells are Pseudomonas bacterial cells.

3. The method of claim 1, wherein said bacterial cells are Pseudomonas aeruginosa bacterial cells.

4. The method of claim 1, wherein said bacterial cells are collected from a biological sample collected from a mammalian subject.

5. The method of claim 4, wherein said biological sample is a blood or tissue sample.

6. The method of claim 4, wherein said biological sample is a sputum or bronchial lavage sample.

7. The method of claim 4, wherein said subject is a human subject.

8. The method of claim 7, wherein said subject is afflicted with cystic fibrosis.

9. The method of claim 1, wherein said step of preparing an extract is carried out with acidified ethyl acetate.

10. The method of claim 1, wherein said step of preparing an extract includes the step of separating said extract by thin layer chromatography.

11. A method of determining the quantity of Psueodomonas present in a biological sample, comprising the steps of:
providing a biological sample collected from a mammalian subject; and then
detecting the quantity of PQS in said biological sample, with the quantity of PQS present in said biological sample directly correlated to the amount of Pseudomonas in said biological sample.

12. The method according to claim 11, wherein said biological sample is a blood or tissue sample.

13. The method according to claim 11, wherein said biological sample is a sputum or bronchial lavage sample.

14. The method according to claim 11, wherein said biological sample is a urine sample.

15. The method of claim 11, wherein said Pseudomonas is P. aeruginosa.

16. The method of claim 11, wherein said detecting step is carried out by exposing said sample or an extract thereof to ultraviolet light and detecting blue fluorescence of PQS in said sample.

17. A method of treating a bacterial infection in a subject in need thereof, comprising administering to said subject a compound of Formula I, or a pharmaceutically acceptable salt thereof, in an amount effective to treat said bacterial infection; 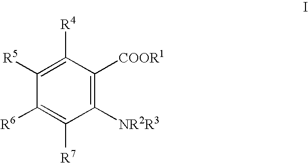
wherein:
R¹ is loweralkyl;
R² and R³ are each independently selected from the group consisting of H and loweralkyl; and
R⁴, R⁵, R⁶, and R⁷ are each independently selected from the group consisting of halo, H, and loweralkyl.

18. The method of claim 17, wherein said infection is a Pseudomonas infection.

19. The method of claim 17, wherein said infection is a Pseudomonas aeruginosa infection.

20. The method of claim 17, wherein R¹ is methyl.

21. The method of claim 17, wherein R² and R³ are both H.

22. The method of claim 17, wherein R⁴, R⁵, R⁶, and R⁷ are H.

23. The method of claim 17, wherein said compound of Formula I is methyl anthranilate.
